(19) United States
(12) Patent Application Publication
(10) Pub. No.: US 2023/0321319 A1
(43) Pub. Date: Oct. 12, 2023

(54) INJECTABLE COMPOSITION AND USE OF SAID COMPOSITION

(71) Applicant: INNATE S.R.L., Novi Ligure (IT)

(72) Inventors: Federico PANZIERI, Novi Ligure (IT); Giulia AVIO, Novi Ligure (IT)

(21) Appl. No.: 18/021,887

(22) PCT Filed: Aug. 13, 2021
(86) PCT No.: PCT/IB2021/057469

§ 371 Date: Feb. 17, 2023

(30) Foreign Application Priority Data
Aug. 18, 2020 (IT) 102020000020224

(51) Int. Cl. A61L 27/02; A61L 27/20

(52) U.S. Cl. CPC A61L 27/20; A61L 27/025; A61L 2400/06; A61L 2430/34

(57) ABSTRACT

Injectable composition in hydrogel form, comprising water and micronized croscarmellose sodium. Method for preparing said injectable composition comprising mixing the components and heat treating the obtained composition at a temperature between 90° C. and 150° C., preferably between 100° C. and 130° C.

The present invention relates to an injectable composition in hydrogel form.

Compositions in hydrogel form are known in the state of the art, adapted to improve the viscosupplementation activity in an intra-articular injection in a joint to increase the lubricating capacities of the synovial fluid of the joint or in an intradermal injection to correct skin imperfections.

However, such compositions either have poor efficacy or are very expensive or difficult to manufacture.

The object of the present invention is to obtain a composition which is inexpensive and simple to produce and which ensures significantly increased viscosupplementation.

Croscarmellose sodium is a not particularly soluble excipient used in the art as a super-disintegrating agent for tablets.

Disintegrating agents, also known simply as disintegrants or disintegrators, have the task of promoting the disintegration and fragmentation of the tablet when it comes into contact with water. Disintegrants are agents added to the tablet and some other encapsulated formulations to promote the breakage of the tablet and capsule assembly into smaller fragments in an aqueous environment, increasing the available surface area and promoting a faster release of the drug.

Tablet disintegration has received considerable attention as an essential step for obtaining rapid drug release. The increasing emphasis on drug availability highlights the importance of the fastest possible disintegration of a tablet as a criterion for ensuring a facilitated drug dissolution behaviour. Disintegrants have the main function of counteracting the efficiency of the tablet binder and the physical forces acting under compression to form the tablet. The stronger the binder, the more effective the disintegrating agents must be for the tablet to release its drugs. Disintegrants are therefore an essential component for tablet formulations and the ability to interact strongly with water is essential for the disintegrating function.

Ideally, the disintegrating agent should cause the fragmentation of the tablet, not only in the granules from which it was compressed, but also in the powder particles from which the granulation was prepared. The disintegrating agent can be added to the compound powder prior to wet granulation; in this case it is referred to as an intragranular disintegrant. Alternatively, it can be added to the already formed granules as a second mixing step, or in both steps. In the first case the disintegration is faster, in the second the tablet is reduced into finer particles.

As for the mechanism of action of the disintegrants, they all act by drawing water into the tablet, or by swelling or creating channels in which the water penetrates by capillarity, thereby promoting the penetration of moisture and the dispersion of the tablet matrix. The disintegrating action therefore originates from the combined effect of swelling, porosity and capillary absorption, deformation and repulsive particle/particle action.

For a long time starch has been the most used disintegrating agent, while in recent years numerous agents known as “super-disintegrants” such as crospovidone, croscarmellose sodium, sodium carboxymethyl amide have been developed.

These new substances are more effective at lower concentrations, with greater disintegrating efficiency and mechanical strength. In contact with water, the super-disintegrants swell, hydrate, change volume or shape and produce a disruptive change in the tablet. Effective super-disintegrants provide improved compressibility, compatibility and have no negative impact on the mechanical strength of formulations containing drugs, even at high doses. The super-disintegrants offer significant improvements with respect to starch.

Croscarmellose sodium is the sodium salt of an internally cross-linked polymer of carboxymethylcellulose. The cross-linking occurs by means of carboxylic ester bonds and reduces solubility in water, while preserving swelling capacity by absorption of water. Croscarmellose sodium can absorb water many times its weight.

US 2019/167844 A1 discloses an injectable hydrogel comprising a homogeneous mixture of cross-linked polymers which penetrate each other in a single step. In one example, such a document discloses the use of carboxymethylcellulose sodium which is cross-linked by means of BDDE (1,4-Butanediol Diglycidyl Ether), a cross-linking agent widely used to stabilize hyaluronic acid-based dermal fillers. Cross-linked hyaluronic acid is then added to the obtained croscarmellose.

However, several publications have drawn attention to the potential adverse health effects of cross-linking by means of BDDE. At the same time, a pure mixture of croscarmellose sodium and hyaluronic acid fails to have satisfactory performance in terms of viscosupplementation or intradermal filling action.

The present invention overcomes the limits set forth above related to the compositions currently known in the state of the art and aims to constitute a functional and advantageous solution both as an embodiment and as a use.

Such objects are obtained, according to the invention, by providing an injectable composition in hydrogel form, comprising micronized croscarmellose sodium.

The present invention has its origin in the surprising discovery that croscarmellose sodium acts in an intra-articular or intradermal injection to improve the viscosification/filling thereof.

The presence of micronized croscarmellose makes the base gel much more viscous, recalling all the water and making it less available after the heat treatment described below, making the entire composition even less subject to degradation.

The use of micronized croscarmellose allows to increase the speed of obtaining the composition in its final form, avoiding the residue of coarse particles.

According to a further exemplary embodiment, the croscarmellose sodium is provided in a weight percentage between 0.01% and 10%.

The composition may further advantageously comprise excipients to ensure the stability and functionality thereof.

In a preferred embodiment, the composition comprises hyaluronic acid.

Hyaluronic acid is a very biocompatible natural polysaccharide, present in all human tissues, and is one of the fundamental components of connective tissues. It is distributed ubiquitously in animal tissues and fluids, in high concentrations in synovial fluids, in the vitreous humour and in the skin, and is mainly responsible for the viscosity and lubricating activity of synovial fluid. In cartilage, hyaluronic acid acts as a support for the aggregation of proteoglycans and proteins.

Hyaluronic acid is a long glycosaminoglycan composed of repeated disaccharides of glucuronic acid and N-acetylglucosamine with high molecular weight and high viscosity.

Hyaluronic acid is currently prepared as a gel from a powder in which it is present as a salt (sodium hyaluronate), and in such a gel it forms water micelles thanks to its high affinity with water itself. A crystalline gel is then formed which, when injected, achieves prolonged durability in tissues, as it maintains biocompatibility.

Hyaluronic acid is used for example in aesthetic medicine to increase the volume of facial tissues, to correct wrinkles, skin folds, to increase the volume of the lips, and in general to correct skin imperfections. In this case it can be injected inside a scar or at the level of the superficial dermal layer for skin treatments and act as a skin moisturiser or even as a filler and therefore anti-wrinkle substance. This action is made possible thanks to the viscoelastic and hydrating properties of hyaluronic acid, which is naturally present in the extracellular matrix of skin with the function of regulating hydration and elasticity. The intradermal administration of the aid allows to give tissues a good amount of hyaluronic acid to counteract the skin aging process.

Alternatively, hyaluronic acid is used for intra-articular administration, in the treatment of osteoarthritis, for example in the knee, where it is naturally found in the synovial fluid, of which it is the main substance responsible for the high lubricating capacities thanks to the viscosity thereof. Hyaluronic acid contributes to lubricating the joint and cushioning mechanical stresses, and therefore has a lubricating function and a cushioning function. It also protects the cartilage from the penetration of inflammatory cells and from the lithic enzymes which degrade it.

In intra-articular infiltrations, the preparation generally consists of a sodium salt of a high molecular weight and high purity fraction of hyaluronic acid. The short half-life of this molecule at the joint level means that its effectiveness is not simply linked to the restoration of physiological levels of hyaluronic acid in osteoarthritic joints. The evaluations carried out in patients with osteoarthritis and other arthropathies involving the knee have shown an action which, in principle, exerts an antiinflammatory activity of the molecule.

Microarthroscopy studies and ultrastructural studies on the joint cartilage and synovial membrane have also shown that there is a possible repair of degenerative joint lesions in humans following intra-articular use of the drug.

Both at the skin level and at the intra-articular level, compositions in hydrogel form with hyaluronic acid aim to achieve a high viscosupplementation to the joint or to the skin. The action of hyaluronic acid and croscarmellose sodium has proved synergistic for the increase of viscosupplementation.

Studies have also shown a significant beneficial influence of the synergistic action of hyaluronic acid and croscarmellose sodium in re-epithelialization, i.e., in that phase of healing in which the granulation tissue consisting of macrophages, fibroblasts and a dense network of newly formed vessels is formed. In this phase, fibroblasts begin to produce collagen and elastin fibres and the epithelium is formed with a slipping mechanism of the trauma-free epithelial cells. Along with this slippage, the epithelial cells begin to reproduce faster. At the same time, the wound contracts, allowing the edges to be brought closer together until they are completely closed. The composition subject of the present invention favours these processes.

In a preferred exemplary embodiment, said hyaluronic acid is linear. This further increases the safety of the composition with respect to health, because there is no need for potentially harmful cross-linking agents in the formation of the hyaluronic acid. Alternatively or in combination, it is possible to use cross-linked hyaluronic acid.

In an embodiment, said croscarmellose sodium is obtained by means of a cross-linking process of carboxymethylcellulose in an acidic environment.

According to a refinement, said acidic environment comprises ethanol, deionized water and hydrochloric acid.

This allows to completely avoid the use of BDDE or other cross-linking agents which are potentially harmful to health.

According to a further embodiment, sodium carboxymethylcellulose is comprised.

The carboxymethylcellulose acts as a gelling agent for the composition. Alternatively or in combination, other gelling substances such as hydroxy ethylcellulose may be used.

According to a further embodiment, sodium hydroxide is comprised.

Sodium hydroxide acts as a pH regulator excipient of the entire formulation to bring it to correct values. Alternatively or in combination, potassium hydroxide may be used.

The action of sodium hydroxide is particularly advantageous in combination with the cross-linking in an acidic environment mentioned above, because it brings the acidic pH of the croscarmellose sodium thus formed back to correct values.

In an embodiment, sodium chloride is comprised. This allows to adjust the osmolarity of the composition.

In a further embodiment, a buffer system is comprised. Such a buffer system may be of any suitable type.

Preferably the buffer system comprises sodium phosphate dibasic and sodium phosphate monobasic.

The buffer system may alternatively be composed of other salts, e.g., potassium equivalents, i.e., potassium phosphate dibasic and potassium phosphate monobasic.

In a further variant, the buffer system may be a citrate buffer, comprising citric acid and sodium citrate.

The buffer can be provided in all the hydration forms thereof.

According to an embodiment, said sodium phosphate dibasic is in the anhydrous and/or dihydrate and/or dodecahydrate hydration state and said sodium phosphate monobasic is in the anhydrous and/or dihydrate hydration state.

Similarly, such hydration states may be provided in the above-mentioned cases of a buffer system comprising potassium salts or a citrate buffer system.

Advantageously, the composition has the appearance of a transparent gel, an important condition for the visual control of syringes.

In a further embodiment, the composition does not include hyaluronic acid and is suitable for veterinary use for the purpose of improving viscosupplementation.

An object of the present invention is furthermore a method for preparing an injectable composition as described above. Such a method includes mixing the components and heat treatment of the composition obtained at a temperature between 90° C. and 150° C., preferably between 100° C. and 130° C. In a preferred embodiment, a gradual heating process of about one hour is used to bring the composition to a temperature of 121° C.

Thanks to this heat-based production methodology, the croscarmellose sodium is made more soluble and able to capture the water molecules around it, creating a very thick and viscous gel.

The micronisation of croscarmellose sodium and the heat treatment of the mixture allow a structural modification of the hydrogel such that a very long-lasting gelatinous layer is formed and it does not swell as usually occurs with injectable hydrogels based on cross-linked hyaluronic acid. Thanks to the cross-linked structural modification induced by the heat treatment, in fact, the composition does not tend to recall water.

The micronisation of the croscarmellose and the heat treatment of the mixture act synergistically as a viscosifying rheological modifier of the hydrogel.

An object of the present invention is also the use of the composition described above in an intra-articular injection to increase the lubricating capacities of the synovial fluid of joints.

An object of the present invention is also the use of the composition described above in an intradermal injection to correct skin imperfections. The composition has a moisturizing effect on the skin and adds firmness, as it acts inside the layers of the dermis, increasing volume and obtaining a corrective effect of skin imperfections.

The aforesaid uses can be envisaged in both the medical and veterinary fields.

These and other features of the invention and the advantages resulting therefrom will become apparent from the following detailed description of an embodiment, preferred among the advantageous and various embodiments of the invention, illustrated merely by way of example, therefore non-limiting, with reference to the table below.

| Component | Weight percentage |
|---|---|
| Water for injections | 93.73 |
| Sodium Chloride | 0.70 |
| Sodium Phosphate Dibasic | 0.30 |
| Sodium Phosphate Monobasic | 0.06 |
| Croscarmellose sodium | 3.00 |
| Carboxymethylcellulose | 1.50 |
| Sodium hyaluronate | 0.50 |
| Sodium hydroxide | 0.21 |

In this embodiment the sodium hyaluronate is preferably low molecular weight, e.g., less than 700 KDa, in particular between 200 and 400 KDa and is preferably in weight percentages less than 3%. Sodium hyaluronate is optionally included.

The croscarmellose sodium is preferably included in micronized form and in weight percentages between 0.01% and 10%.

Sodium chloride is optionally included, e.g., in weight percentages between 0% and 4%.

Sodium phosphate dibasic is optionally included, e.g., in weight percentages between 0% and 5%. The sodium phosphate dibasic is preferably in the dodecahydrate hydration state.

Sodium phosphate monobasic is optionally included, e.g., in weight percentages between 0% and 5%. The sodium phosphate monobasic is preferably in the hydrated state.

Sodium carboxymethylcellulose is optionally included, e.g., in weight percentages between 0% and 10%.

Sodium hydroxide is optionally included, e.g., in weight percentages between 0% and 6% in a 30% solution.

In a further embodiment, the composition having the formulation illustrated in the table is suitable for veterinary use for the purpose of improving viscosupplementation. In this case, the composition preferably does not include hyaluronic acid.

From the foregoing, it is therefore evident that the invention is not limited to the embodiments just described and illustrated by way of non-limiting examples, but may be varied and modified, as a whole and in individual details, especially constructively, according to the specific needs and conveniences of production and use, within the scope of the technical and functional equivalents, without abandoning the guiding principle set forth above and subsequently claimed.

1. An injectable composition in hydrogel form comprising croscarmellose sodium,
characterized in that
said croscarmellose sodium is micronized.

2. The injectable composition according to claim 1,
wherein the croscarmellose sodium is provided in weight percentages between 0.01% and 10%.

3. The injectable composition according to claim 1, further comprising hyaluronic acid.

4. The injectable composition according to claim 3,
wherein said hyaluronic acid is linear.

5. The injectable composition according to claim 1, further comprising carboxymethylcellulose sodium .

6. The injectable composition according to claim 1, further comprising sodium hydroxide.

7. The injectable composition according to claim 1, wherein sodium chloride is comprised.

8. The injectable composition according to claim 1, further comprising a buffer system, said buffer system comprising sodium phosphate dibasic and sodium phosphate monobasic, wherein said sodium phosphate dibasic is in the anhydrous and/or dihydrate and/or dodecahydrate hydration state and said sodium phosphate monobasic is in the anhydrous and/or dihydrate hydration state.

9. A method for preparing the injectable composition according to claim 1,
characterized in that
it includes mixing the-components and heat treatment of the composition obtained at a temperature between 90° C. and 150° C., preferably between 100° C. and 130° C.

10. The method according to claim 9, wherein said croscarmellose sodium is obtained by a carboxymethylcellulose cross-linking process in an acidic environment.

11. The method according to claim 10, wherein said acidic environment comprises ethanol, deionized water, and hydrochloric acid.

12. A method of use of the composition according to claim 1, comprising using the composition in an intra-articular injection in a joint to increase lubricating capacities of the-synovial fluid of the joint.

13. A method of use of the composition according to claim 1, comprising using the composition in an intradermal injection to correct skin imperfections.
